(19) United States
(12) Patent Application Publication
(10) Pub. No.: US 2011/0288359 A1
(43) Pub. Date: Nov. 24, 2011

(54) PROCESS FOR PREPARATION OF A CATIONIC ZEOLITE BY ION EXCHANGE

(75) Inventors: Karin Barthelet, Lyon (FR); Patrick Magnoux, Poitiers (FR); Alain Methivier, Lyon (FR); Vania Santos-Moreau, Lyon (FR)

(73) Assignee: IFP Energies nouvelles, RUEIL-MALMAISON Cedex (FR)

(21) Appl. No.: 12/994,223

(22) PCT Filed: May 5, 2009
(86) PCT No.: PCT/FR2009/000528

§ 371 Date: Jan. 28, 2011

(30) Foreign Application Priority Data
May 28, 2008 (FR) 08/02.949

(51) Int. Cl. C07C 7/20; C01B 39/24; C01B 39/22; C01B 39/00; C01B 39/14

(52) U.S. Cl. 585/852; 423/700

(57) ABSTRACT

The invention relates to a process for preparation of a cationic zeolite that is at least partially exchanged with one or more monovalent and/or multivalent cations. The process comprises at least the stages for separate activation of a zeolite that is to be exchanged and an anhydrous salt under a dry, inert gaseous stream, dissolution of the anhydrous salt in an anhydrous organic solvent, ion exchange under dry inert atmosphere, filtering and washing with an anhydrous organic solvent, and calcination in the presence of oxygen and under a dry gaseous stream. The invention also relates to the use of prepared zeolites for the separation or the purification of hydrocarbon feedstocks.

### PRIOR ART

Among the processes for separation or purification, there are quite a number that use zeolites in cationic as well as adsorbent form. Their principle resides either in a selectivity of shape or size, or in a particular affinity of one of the components of the feedstock for the cations. The latter are to be avoided to the extent that the process does not involve any chemical reaction. Any transformation of compounds of the feedstock actually leads to a reduction in yield and can also be at the origin of the formation of coke precursors, thus producing a premature aging of the adsorbent. These undesirable phenomena are all the more frequent as the zeolite has active surface sites that are most often acid sites. Therefore, contrary to the protonated zeolites, the cationic zeolites that do not have a priori Brönsted acid sites should not have strong activity. Nevertheless, in some cases, they have non-negligible activities that are characterized by reactions that involve acid sites.

To be able to obtain zeolites that are very sparingly reactive or even non-reactive, it is necessary either to neutralize the detected activity or to prevent its presence. The neutralization in particular with basic solutions is not always effective and exhibits the drawback of adding a stage to the preparation of the zeolite. The second option would therefore be preferable. One solution that was already proposed in the literature is to activate the solids under reducing atmosphere such as NH₃ (H. Siegel, R. Schöllner, B. Staudte, J. J. Van Dun, W. J. Mortier, Zeolites, 1987, 7, 372) so as to be able to neutralize the protons as soon as they are formed. However, the thus obtained zeolites are able to contain NH₃ molecules and therefore to no longer have their entire porosity accessible.

To remedy this, a process for preparation by ion exchange of a cationic zeolite that is exchanged at least partially with one or more monovalent and/or multivalent cations that proves effective for limiting and even cancelling the reactivity of these zeolites is proposed within the framework of this invention. The zeolites according to the invention are less active with regard to reactions that involve acid sites than when they are prepared according to the prior art.

### SUMMARY DESCRIPTION OF THE INVENTION

The invention relates to a process for preparation of a cationic zeolite that is at least partially exchanged with one or more monovalent and/or multivalent cations. The process comprises at least the stages for separate activation of a zeolite that is to be exchanged and an anhydrous salt under a dry, inert gaseous stream, dissolution of the anhydrous salt in an anhydrous organic solvent, ion exchange under dry inert atmosphere, filtering and washing with an anhydrous organic solvent, and calcination in the presence of oxygen and under a dry gaseous stream. The invention also relates to the use of prepared zeolites for the separation or the purification of hydrocarbon feedstocks.

### DETAILED DESCRIPTION OF THE INVENTION

The invention relates to a process for preparation of a cationic zeolite that is at least partially exchanged with one or more monovalent and/or multivalent cations, whereby said exchanged cationic zeolite is preferably of type X, Y, A, β, or MFI, whereby said preparation process comprises at least the following stages:
a) Separate activation of a zeolite that is to be exchanged and an anhydrous salt under a dry, inert gaseous stream,
b) Dissolution of the anhydrous salt, obtained at the end of stage a), in an anhydrous organic solvent, preferably an anhydrous alcohol, in a very preferred manner anhydrous ethanol,
c) Ion exchange by adding the activated zeolite that is obtained at the end of stage a) in the solution that is obtained at the end of stage b), whereby this stage is implemented under dry inert atmosphere,
d) Filtering and washing the solid that is obtained at the end of stage c) with an anhydrous organic solvent,
e) Calcination of the solid that is obtained at the end of stage d) in the presence of oxygen and under a dry gaseous stream.

The multivalent cation(s) is/are generally divalent or trivalent cations and are generally alkaline-earth cations or lanthanides. The monovalent cation(s) is/are generally alkaline cations.

The stages a), b), e), d) and e) of the preparation process can generally be implemented under the operating conditions described below.

### Stage a)

The separate activation of the zeolite that is to be exchanged and the anhydrous salt is preferably implemented in two separate columns.

The zeolite is generally activated under a dry, inert gaseous stream, preferably under a dry nitrogen stream, encompassed between 3 and 8 l·h⁻¹·g⁻¹ and at a temperature of between 200 and 600° C., preferably between 350° C. and 550° C., and in a very preferred manner between 450 and 550° C., for a time period of between 1 and 20 hours.

The anhydrous salt is generally activated under a dry, inert gaseous stream, preferably under a dry nitrogen stream, encompassed between 3 and 8 l·h⁻¹·g⁻¹ and at a temperature of between 50 and 350° C., and for a time period of between 1 and 20 hours.

### Stage b)

Stage b) for dissolution of the anhydrous salt that is obtained at the end of stage a) in the anhydrous organic solvent in which a dry inert gas, preferably dry argon, is bubbled is generally implemented at a temperature of between 20 and 60° C. while being stirred magnetically at a speed of between 500 and 700 rpm.

### Stage c)

Stage c) for ion exchange by adding the activated zeolite that is obtained at the end of stage a) in, the solution that is obtained at the end of stage b) is generally implemented while being stirred at between 500 and 700 rpm, and at a temperature of between 20 and 100° C., whereby this stage is implemented under dry inert atmosphere, preferably dry argon, whereby the time period of the exchange is between 1 and 20 hours.

A cooling system is generally adapted so as to prevent the evaporation of the anhydrous organic solvent during the exchange.

### Stage d)

Stage d) is that of filtering and washing the solid that is obtained at the end of stage c) with an anhydrous organic solvent, preferably an anhydrous alcohol, and in a very preferred manner anhydrous ethanol.

The volume of the anhydrous organic solvent that is used is in general at least equal to the one that is used during the ion exchange stage.

Furthermore, the solid that is obtained at the end of stage d) can be stored without running the risk of its acido-basic characteristics changing.

### Stage e)

Stage e) for calcination of the solid that is obtained at the end of stage d) in the presence of oxygen is generally implemented at a temperature of between 200 and 600° C., preferably between 300 and 550° C., for a time period of between 1 and 20 hours, preferably between 10 and 15 hours, under a dry gaseous stream that is between 3 and 8 l·h⁻¹·g⁻¹, and preferably under a stream of dry compressed air.

It is generally verified by adsorption of nitrogen at 77° K that the zeolite has preserved its pore volume.

The exchange rate that is obtained in the monovalent and/or multivalent cation(s) is generally verified by Inductively Coupled Plasma Atomic Emission Spectrometry (ICP-AES).

The thus prepared cationic zeolite can be used in any process for separation or purification of hydrocarbon feedstocks. Among other potential applications, it is possible to cite the separation of paraxylene from an aromatic C8 fraction, the separation of linear paraffins from a kerosene fraction, the separation of linear paraffins/branched paraffins from a gasoline fraction, the separation of paraffins/olefins, the elimination of mercaptans from natural gas, the desulfurization of the FCC gasolines, and the denitration of C4-C6 feedstocks for oligomerization.

The reduction in activity can generally be demonstrated by testing the zeolite using a model reaction that involves the isomerization of 1-dodecene. Its principle, its implementation, and its exploitation are explained in the literature (V. Santos, K. Barthelet, I. Gener., C. Canaff, P. Magnoux, Microporous and Mesoporous Materials, in press).

The implementation of this test, which is a batch and liquid-phase experiment, comprises the following stages:
a) Suspending 3 g of zeolite in 75 g of 95% pure 1-dodecene, provided by Aldrich. This suspension is produced in a three-neck flask.
b) Degassing the suspension that is prepared in a) for several minutes by bubbling argon, and then holding the latter under inert argon atmosphere but without bubbling.
c) Putting under magnetic stirring at 500 rpm and under heating in a silicone oil bath at 150° C. for 24 hours.
The three-neck flask is topped with a reflux cooling system for preventing any evaporation of solution during the experiment.
d) Samples of 0.05 ml taken at regular time intervals and analysis of the latter by gas phase chromatography on a PONA (paraffins, olefins, naphthenes, aromatic compounds) column with a diameter of 200 μm, 0.5 μm film thickness, and 50 m length.

Starting from the chromatograms, the compositions of each sample of 1-dodecene and its different isomers (2-dodecene, 3-dodecene, 4-dodecene, 5-dodecene and 6-dodecene) are determined from which the conversion of 1-dodecene is calculated according to the following equation:

$$Conv ( 1 - dodecene ) = \frac{{n ( 1 - dodecene )}_{initial} - {n ( 1 - dodecene )}_{final}}{{n ( 1 - dodecene )}_{initial}} \times 100.$$

It is then possible to trace the curve of the conversion into 1-dodecene based on the reaction time, the slope of the tangent to the first points of this curve corresponding to the initial speed of the isomerization reaction of 1-dodecene and reflecting the initial activity of the tested zeolite, i.e., its number of active sites relative to a reaction that involves acid sites.

### EXAMPLES

Two NaCaY zeolites with an approximately 25% exchange rate are prepared by ion exchange starting from an NaY zeolite in powder form. One of the two NaCaY zeolites is prepared in an aqueous medium, and the other is prepared in an anhydrous alcoholic medium.

For the exchange in aqueous medium, approximately 10 g of zeolite is put directly in suspension in 1 l of 0.7 g/l of CaCl₂ solution (solution prepared starting from CaCl₂.2H₂O from Aldrich). Then, the flask is heated using a 60° C. silicone bath, and the suspension is kept under magnetic stirring. A cooling system is adapted so as to prevent the evaporation of the suspension during the exchange. The exchange lasts for approximately 7 hours.

After the exchange, the zeolite is filtered, washed with water, and dried in an oven at 110° C. Then, it is dehydrated under a stream of nitrogen (3 l·h⁻¹·g_{zeol}⁻¹) in a tubular furnace at 450° C. for 12 hours so as to eliminate the water that is adsorbed in the zeolite during the exchange. The thus prepared zeolite is referred to as NaCaY-26% (aqueous medium).

For the alcoholic medium exchange, the procedure that is described in the description of the invention is applied. Before the exchange itself, the zeolite and the salt are dehydrated separately. The NaY zeolite is dehydrated under a nitrogen stream (3 l·h⁻¹·g_{zeol}⁻¹) for 12 hours at 450° C. in a tubular furnace, and the anhydrous CaCl₂ salt of Aldrich is dehydrated under a nitrogen stream for 12 hours at 150° C. in a column in a tubular furnace. The two columns—one containing the dehydrated zeolite and the other containing the dehydrated salt—are isolated before being disconnected from the furnaces to preserve them from all contact with air.

The dehydrated salt is dissolved, while being stirred magnetically at 500 rpm, in anhydrous ethanol in which argon is bubbled in a round-bottomed flask. The zeolite is then added to this solution (10 g of solid per 1 l of solution). Then, the flask is heated using a 60° C. silicone bath, and the suspension is kept under magnetic stirring at 500 rpm. A cooling system is adapted so as to prevent the evaporation of the alcohol during the exchange. The system is kept under argon to prevent any contact of the suspension with the air. The exchange lasts for approximately 7 hours.

After the exchange, the solid is filtered and Washed with anhydrous ethanol (same volume as the one used during the ion exchange). The solid that is recovered is calcined in a column in a tubular furnace at 550° C. for 20 hours under a flow of dry compressed air. The thus prepared zeolite is referred to as NaCaY-24% (anhydrous ethanol medium).

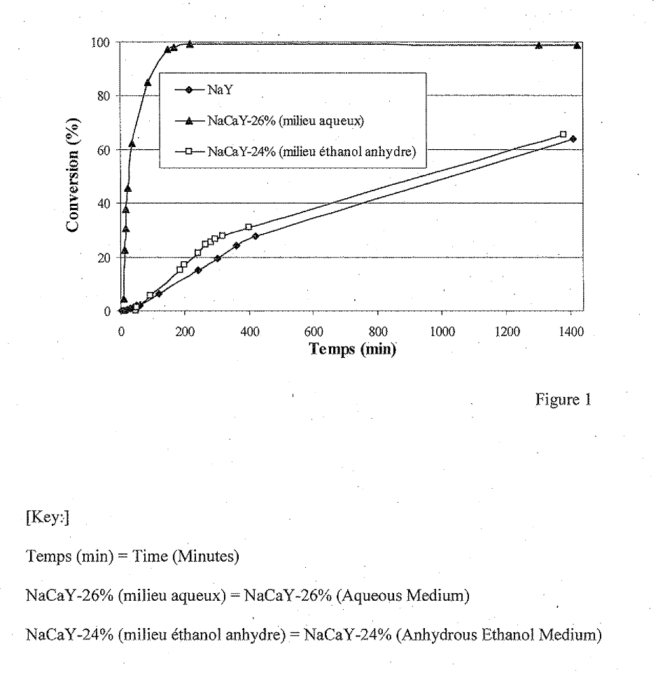

The residual acidity of these three solids is determined via a model transformation reaction of an olefin (1-dodecene) that makes it possible to characterize the low activities. The differences in activity of the two NaCaY between one another and with the starting NaY are presented in FIG. 1, which shows the variation of the conversion of the 1-dodecene as a function of time for NaY, NaCaY-26% (aqueous medium) and NaCaY-24% (anhydrous ethanol medium).

| | | End-of-Conver- | Number of |
|---|---|---|---|
| | Initial Speed | sion Test | Isomers |
| Solid | (mol · h⁻¹ · g_{zeol}⁻¹) | (24 hours) % | Formed |
| NaY | 6 · 18 · 10⁻³ | 64 | 4 |
| NaCaY-26% | 1 · 28 · 10⁻¹ | 100 | 10 |
| (Aqueous Medium) | | | |
| NaCaY-24% | 9 · 84 · 10⁻³ | 65 | 4 |
| (Anhydrous Ethanol | | | |
| Medium) | | | |

It can be noted that the activity of the NaCaY solid is greatly reduced when it is prepared in alcoholic medium and not in aqueous medium and becomes similar to that of the starting NaY (initial reaction speeds, conversion and number of products formed-very close).

1. Process for preparation of a cationic zeolite that is at least partially exchanged with one or more monovalent and/or multivalent cations, whereby said preparation process comprises at least the following stages:
a) Separate activation of a zeolite that is to be exchanged and an anhydrous salt under a dry, inert gaseous stream,
b) Dissolution of the anhydrous salt, obtained at the end of stage a), in an anhydrous organic solvent,
c) Ion exchange by adding the activated zeolite that is obtained at the end of stage a) in the solution that is obtained at the end of stage b), whereby this stage is implemented under dry inert atmosphere,
d) Filtering and washing the solid that is obtained at the end of stage c) with an anhydrous organic solvent,
e) Calcination of the solid that is obtained at the end of stage d) in the presence of oxygen and under a dry gaseous stream.

2. Process for preparation according to claim 1, in which the anhydrous organic solvent is an anhydrous alcohol.

3. Process for preparation according to claim 1, in which the anhydrous organic solvent is anhydrous ethanol.

4. A process for preparation according to claim 1, wherein the at least partial exchange is with multivalent cation(s) which is/are alkaline-earth cations or lanthanides.

5. A process for preparation according to claim 1, wherein the at least partial exchange is with monovalent cation(s) which is/are alkaline cations.

6. A process for preparation according to claim 1, in which the exchanged cationic zeolite is of type X, Y, A, β, or MPI.

7. A process for preparation according to claim 1, in which stages a), b), c), d) and e) are implemented under the following operating conditions:
a) Separate activation of a zeolite that is to be exchanged and the anhydrous salt, whereby said zeolite is activated under a dry, inert gaseous stream that is encompassed between 3 and 8 l·h⁻¹·g⁻¹ and at a temperature of between 200 and 600° C., for a time period of between 1 and 20 hours, whereby said anhydrous salt is activated under a dry, inert gaseous stream that is encompassed between 3 and 8 l·h⁻¹·g⁻¹ and at a temperature of between 50 and 350° C., for a time period of between 1 and 20 hours,
b) Dissolution of the anhydrous salt, obtained at the end of stage a) in the anhydrous organic solvent in which a dry inert gas is bubbled, whereby this stage is implemented at a temperature of between 20 and 60° C. while being stirred magnetically at a speed of between 500 and 700 rpm,
c) Ion exchange by adding the activated zeolite that is obtained at the end of stage a) in the solution that is obtained at the end of stage b), whereby this stage is implemented while being stirred between 500 and 700 rpm at a temperature that is between 20 and 100° C., whereby this stage is implemented under dry inert atmosphere, with the time period of the exchange being between 1 and 20 hours,
d) Filtering and washing the solid that is obtained at the end of stage e) with an anhydrous organic solvent,
e) Calcination of the solid that is obtained at the end of stage d) in the presence of oxygen at a temperature of between 200 and 600° C., for a time period of between 1 and 20 hours, and under a dry gaseous stream that is encompassed between 3 and 8 l·h⁻¹·g⁻¹.

8. A process comprising separation or purification of hydrocarbon feedstocks by contacting said hydrocarbon feedstocks with the prepared zeolites according to claim 1.

9. A process according to claim 8 comprising the separation of paraxylene from a C8 fraction.

10. A process according to claim 8 comprising the separation of linear paraffins from a kerosene fraction.

11. A process according to claim 8 comprising the separation of linear paraffins/branched paraffins.

12. A process according to claim 8 comprising the separation of paraffins/olefins.

13. A process according to claim 8 comprising the elimination of mercaptans from natural gas.

14. A process according to claim 8 comprising the desulfurization of FCC gasolines.

15. A process according to claim 8 comprising the denitration of feedstocks for oligomerization.
